(19) United States
(12) Patent Application Publication
(10) Pub. No.: US 2025/0040919 A1
(43) Pub. Date: Feb. 6, 2025

(54) DRAINAGE DEVICES, SYSTEMS, AND METHODS

(71) Applicant: BOSTON SCIENTIFIC SCIMED, INC., Maple Grove, MN (US)

(72) Inventors: Paul Smith, Smithfield, RI (US); Yeison Calvo, San Antonio (CR); Gian Franco Loo Fuchs, San Jose (CR); Gonzalo Jose Saenz Villalobos, Alajuela (CR); Peter L. Dayton, Brookline, MA (US)

(73) Assignee: BOSTON SCIENTIFIC SCIMED, INC., Maple Grove, MN (US)

(21) Appl. No.: 18/790,672

(22) Filed: Jul. 31, 2024

Related U.S. Application Data
(60) Provisional application No. 63/517,035, filed on Aug. 1, 2023.

(51) Int. Cl. A61B 17/00

(52) U.S. Cl. CPC A61B 17/0057; A61B 2017/00575; A61B 2017/00579; A61B 2017/00592; A61B 2017/00601; A61B 2017/00623; A61B 2017/00628; A61B 2017/00641; A61B 2017/00654

(57) ABSTRACT

Devices, systems, and methods for treating defects in anatomical structures are disclosed. A device may include a first portion configured to engage body tissue and a second portion extending distally from the first portion. The second portion may be elongated and configured to extend into and/or engage tissue of a defect in an anatomical structure. The device may have an expanded or deployed configuration in which at least the second portion of the device has a first diameter. The device may be configured to be adjusted to a closed configuration in which the second portion of the device has a second diameter that is reduced relative to the first diameter. The second portion of the device may include one or more tissue engaging elements configured to engage the defect such that adjusting the second portion to the reduced second diameter closes the defect.

### CROSS-REFERENCE TO RELATED APPLICATIONS

This application claims the benefit of U.S. Provisional Patent Application Ser. No. 63/517,035, filed Aug. 1, 2023, which is incorporated herein by reference.

### TECHNICAL FIELD

The present disclosure pertains generally, but not by way of limitation, to defect treatment devices and systems, and methods of treatment. More particularly, the present disclosure relates to devices, systems, and methods for use in closing defects in anatomical structures (e.g., body lumens, body passages, body cavities, etc.) of a patient.

### BACKGROUND

A wide variety of intracorporeal and extracorporeal medical devices and systems have been developed for medical use, for example, in endoscopic procedures. Some of these devices and systems include guidewires, catheters, catheter systems, endoscopic instruments, wound or defect treatment devices, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices.

In a first example, a device for implantation in an anatomical defect at or through body tissue may include a first portion configured to engage the body tissue and a second portion extending distally from the first portion, the second portion is configured to engage tissue of the anatomical defect, the anatomical defect having a first opening, a second opening, and a passage extending between the first opening and the second opening, and wherein the second portion may be configured to be reduced in diameter while engaging the tissue of the anatomical defect to close the anatomical defect.

Additionally or alternatively to any of the examples above, the second portion may be tubular.

Additionally or alternatively to any of the examples above, the device may include one or more barbs extending from the second portion, the one or more barbs are configured to engage the tissue of the anatomical defect.

Additionally or alternatively to any of the examples above, the one or more barbs may extend in a circumferential direction around the second portion and are configured to engage the tissue of the anatomical defect in response to rotation of the second portion.

Additionally or alternatively to any of the examples above, the device may include a first set of one or more barbs extending from the first portion in a first circumferential direction, the first set of one or more barbs are configured to engage the body tissue and a second set of one or more barbs extending from the second portion in a second circumferential direction, the second set of one or more barbs are configured to engage the tissue of the anatomical defect.

Additionally or alternatively to any of the examples above, the second set of one or more barbs may be configured to engage the tissue of the anatomical defect in response to rotation of the second portion in the second circumferential direction, and while engaging the tissue of the anatomical defect, the second portion may be configured to be reduced in diameter to close the anatomical defect in response to rotation of the first portion in the second circumferential direction.

Additionally or alternatively to any of the examples above, the first set of one or more barbs may be configured to engage the body tissue to prevent rotation of the first portion and the second portion in the first circumferential direction.

Additionally or alternatively to any of the examples above, the device may include a first set of one or more barbs extending from the first portion, the first set of one or more barbs are configured to engage the body tissue and a second set of one or more barbs extending from the second portion, the second set of one or more barbs are configured to engage tissue distal of the anatomical defect.

Additionally or alternatively to any of the examples above, the device may include a tool for engaging the first portion, and wherein the first portion may have an opening for receiving a distal end of the tool and the tool is configured to be rotated when received within the opening to reduce the diameter of the second portion.

Additionally or alternatively to any of the examples above, the device may include a cord wrapped about the second portion and extending proximal of the first portion, wherein the cord may cause the second portion to be reduced in diameter in response to a tension applied to the cord.

Additionally or alternatively to any of the examples above, the device may include a clip, wherein the clip is configured to be secured to the cord at a location proximate the first portion when a tension is applied to the cord to maintain the tension on the cord.

Additionally or alternatively to any of the examples above, the first portion may include a wedge configured to receive the cord after the tension is applied to the cord and maintain the tension on the cord.

Additionally or alternatively to any of the examples above, the first portion and the second portion may be formed from one or more interwoven wires.

In another example, a system for implanting an implantable device in an anatomical defect at or through body tissue may include an elongate tube configured for insertion to the body tissue proximate the anatomical defect, an deployment device configured to be received within the elongate tube, and a implantable medical device configured to be received within the elongate tube and engage the deployment device, the implantable medical device comprising: a first portion configured to engage the body tissue, and a second portion extending distally from the first portion, the second portion is configured to engage tissue of the anatomical defect, the anatomical defect having a first opening, a second opening, and a passage extending between the first opening and the second opening, and wherein the deployment device may be configured to be actuated to advance the second portion of the implantable medical device out of the elongate tube to a deployed configuration having a first diameter within the anatomical defect and reduce a diameter of the second portion of the implantable medical device within the anatomical defect to a second diameter.

Additionally or alternatively to any of the examples above, the deployment device may be configured to engage and rotate the first portion of the implantable medical device to reduce the diameter of the second portion of the implantable device.

Additionally or alternatively to any of the examples above, the implantable medical device may comprise a cord wrapped about the second portion, and the deployment device may be configured to engage the cord and apply a tension to the cord to reduce the diameter of the second portion of the implantable medical device to the second diameter.

In another example, a method of implanting an implantable medical device in an anatomical defect at or through body tissue may include positioning an elongate portion of the implantable medical device within the anatomical defect, closing the anatomical defect in response to reducing a diameter of the implantable medical device within the anatomical defect from a first diameter to a second diameter, and securing the implantable medical device at or proximate the anatomical defect in a configuration having the second diameter.

Additionally or alternatively to any of the examples above, closing the anatomical defect in response to reducing the diameter of the elongate portion may comprise engaging one or more barbs extending from the elongate portion of the implantable medical device with tissue of or proximate the anatomical defect.

Additionally or alternatively to any of the examples above, closing the anatomical defect in response to reducing the diameter of the elongate portion may comprise applying a tension to a cord wrapped about the elongate portion.

Additionally or alternatively to any of the examples above, securing the implantable medical device at or proximate the anatomical defect in the configuration having the second diameter may comprise engaging barbs extending from the implantable medical device with the body tissue.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIGS. 1A and 1B are schematic views illustrating an implantation of an illustrative device in a defect through body tissue;

FIG. 2 is a schematic view of an illustrative device for implanting in a defect through body tissue;

FIGS. 3A and 3B are schematic views of an illustrative device for implanting in a defect through body tissue, where a portion of the illustrative device transitions between a first diameter and a second diameter;

FIG. 4 is a schematic view of an illustrative system for implanting a device in a defect through body tissue;

FIG. 5 is a schematic view of an illustrative device for implanting in a defect through body tissue;

FIG. 6 is a schematic view of an illustrative device for implanting in a defect through body tissue;

FIG. 7 is a schematic view of an illustrative device for implanting in a defect through body tissue tract;

FIGS. 8A-8D are schematic views depicting an implantation of an illustrative device in a defect through body tissue; and

FIGS. 9A-9C are schematic views depicting an implantation of an illustrative device in a defect through body tissue.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Defects in patient tissue may form openings or connections in or through one or more anatomical structures. The defects may occur naturally and/or as a result of an injury, clinical procedure, surgery, or infection. The defects referred to herein may be anatomical defects, which are defects of an anatomical structure, such as a fistula between anatomical structures formed naturally or surgically and/or an opening into or passage through a wall of an anatomical structure formed naturally or surgically. In some examples, anatomical defects may have a first open end, a second open end, and a passage between the first open end and the second open end, where the passage may be elongated or not (e.g., where the anatomical defect may be an opening at or through body tissue of an anatomical structure that allows leakage), but other suitable anatomical defects are contemplated. Some example anatomical defects may include, but are not limited to, fistulas, which are abnormal openings through or connections between organs, blood vessels, and/or other body structures, gastro-gastric fistulas, diverticulosis pouches, perforations, and/or other defects in body tissue. Such defects may occur at or affect a variety of body parts including, but not limited to, body parts of the GI tract (e.g., the upper GI tract and/or the lower GI tract), the stomach, the intestines, the bowels, body parts of the reproductive tract, the uterus, and/or other body parts.

The body parts may have body tissue defining body cavities or passages of the body part in the absence of a defect. Tissue of a defect may be tissue of a body part that defines the defect.

When the defects form openings, pouches, or connections in or through one or more body parts, it may be critical to proactively close the defects to avoid migration of material or fluids to body structures not intended to receive the material or fluids as the defects do not typically heal without intervention. If the defects are not closed, the defects may increase in size and may eventually cause cancer and/or other diseases.

Defects forming openings or connections in or through one or more body parts may be closed using an endoscopic procedure. In some examples, a device (e.g., an implantable medical device) for implantation in or at an anatomical defect may be or may include a stent device deliverable to a defect during an endoscopic procedure.

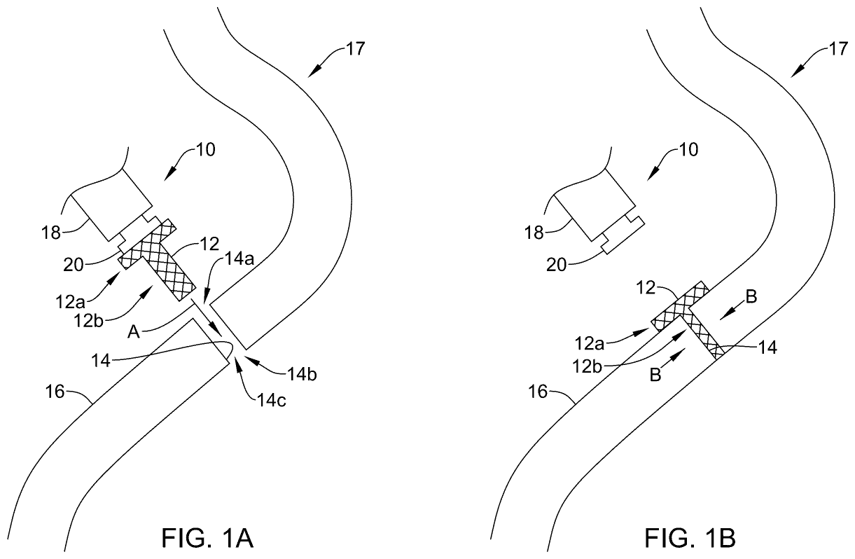

FIGS. 1A and 1B depict schematic views of a system 10 including an implantable device 12 (e.g., an implantable medical device), where the system 10 may be configured to facilitate implantation of the implantable device 12 in a defect 14 (e.g., an anatomical defect) at or through body tissue 16 of a body part 17. The system 10 may include an elongate tube 18 (e.g., a catheter, a sheath, etc.), a deployment device 20, the implantable device 12, and/or one or more other suitable components.

The elongate tube 18 may be a catheter, a sheath, and/or other elongate tube suitable for delivering the implantable device 12 to a defect (e.g., the defect 14 or other suitable defect) at, in, or affecting a body part 17. In some cases, the elongate tube 18 may be configured to receive the deployment device 20 and the implantable device 12 for delivery to the defect 14.

The deployment device 20 may be any elongate component or components configured to be at least partially received within the elongate tube 18 and to engage the device 12 to deploy the device 12 from the elongate tube 18. The deployment device 20 may be and/or may include a shaft, a tube, wire, and/or other elongated component.

The deployment device 20 may be configured to engage and/or grasp a proximal or first portion 12a of the device 12. In some examples, the deployment device 20 may threadedly engage the device 12, may have a keyed relationship with the proximal portion of the device 12, may clasp the device 12, and/or may otherwise be configured to engage and/or grasp the proximal or first portion 12a of the device 12.

The deployment device 20 may be configured to move relative to the elongate tube 18. In some examples, the deployment device 20 may be configured to translate longitudinally or axially relative to the elongate tube 18 to advance and/or deploy the device 12 out of the elongate tube. Additionally or alternatively, the deployment device 20 may be configured to rotate about a central axis relative to the elongate tube 18 to rotate the device 12, which may close a passage (e.g., a lumen, opening, etc.) through the device 12 and thus, close the defect 14.

As depicted in FIG. 1A, the device 12 may be delivered toward the defect 14 at or through the body tissue 16. Although other suitable defects are contemplated, the defect 14 may include a first open end or opening 14a, a second open end or opening 14b, and a passage 14c extending between the first opening 14a and the second opening 14b. The device 12 may include the first portion 12a and a second portion 12b, where the second portion 12b may be elongated and may extend distally from the first portion 12a in a manner configured to be advanced into the defect 14 in the direction of arrow A.

The device 12 may be configured to adjust between one or more sizes (e.g., diameters), including, but not limited to, a size associated with a delivery configuration, a size associated with an expanded or deployed configuration (e.g., as depicted in FIG. 1A), and a size associated with a closed configuration (e.g., as depicted in FIG. 1B). In the delivery configuration, the first portion 12a and the second portion 12b of the device may be constrained or restrained while being delivered within the elongate tube 18 to the defect 14. In the deployed configuration, at least the second portion 12b of the device 12 may be advanced out of the elongate tube 18 and expanded to engage an inner wall of the defect 14 upon insertion into the defect 14, while the first portion 12a of the device 12 may remain constrained or restrained within the elongate tube 18. Alternatively, in the deployed configuration, the first portion 12a and the second portion 12b of the device 12 may be advanced out of the elongate tube 18 and expanded, where the second portion 12b may be positioned within the defect 14 and the first portion may be at or proximal of the defect 14 to act as a barrier to fluid or material flowing into the defect 14, as depicted in FIG. 1A.

Once the device 12 is positioned within the defect 14, the second portion 12b of the device 12 may engage tissue of the defect 14 and may be reduced in diameter to close the defect 12. In some examples, the second portion 12b of the device 12 may be reduced in diameter from a first diameter associated with the deployed configuration to a second diameter associated with the closed configuration. If the first portion 12a of the device 12 was not deployed or advanced out of the elongate tube 18 prior to closing the defect with the second portion 12b of the device 12, then the first portion 12a may be advanced out of the elongate tube 18 and expanded after closing the defect 14 to act as a further barrier to fluid or material flow into or through the defect 14.

The second portion 12b of the device 12 may be reduced in diameter using any suitable technique including, but not limited to, applying a longitudinal tension to the device 12, applying a tension to a cord wrapped around (e.g., helically wrapped around) the second portion 12b of the device 12, rotating the first portion 12a of the device to twist the second portion 12b, and/or through one or more other suitable techniques.

The device 12 may be configured in any suitable manner. In some examples, a body of the device 12 may be formed from one or more interwoven wires (e.g., metal wires, polymer wires, threads, strands, filaments, etc.), braided wires, a cut tube, and/or formed in one or more other suitable manners. Further, the device 12 may be a stent or may be a stent-like structure configured to be collapsed during implantation, but this is not required.

The body of the device 12 may be formed from any suitable materials. For example, the interwoven wires, cut tube, and/or other components of the body of the device 12 may be formed from a metallic material, a polymer material, a combination of metallic materials and polymer materials, a nickel-titanium alloy (e.g., Nitinol™), a shape-memory material, and/or other suitable materials.

One or more layers of biocompatible materials (e.g., coatings, sheets or sheet-like materials, etc.) may be applied to an outer surface and/or an inner surface of the device 12. In some cases, the biocompatible material may be configured to promote growth of tissue at or about the defect 14, but this is not required.

The device 12 may be heat-shaped and/or shaped in one or more other suitable manners that biases the material of the device 12 a pre-set shape. When the shape of the device 12 in the delivery configuration and the shape of the device 12 in the deployed configuration are not the same, the shaped device 12 may automatically adjust from (e.g., biased from) a delivery configuration to a deployed configuration upon being deployed or advanced out of the elongate tube 18. In some cases, the device 12 may be constrained when in the delivery configuration and fully relaxed or at least partially relaxed when in the deployed configuration.

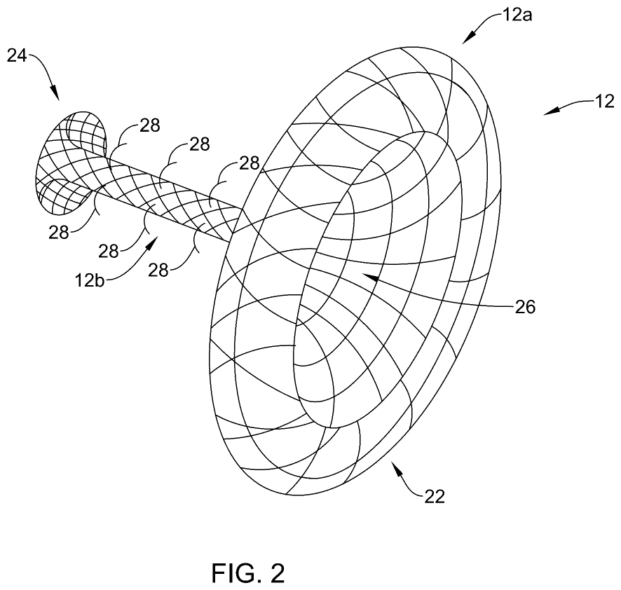

FIG. 2 depicts a schematic perspective view of an illustrative device 12 in the deployed configuration, where the device 12 may include the first portion 12a at a proximal end 22, a distal end 24, and the second portion 12b extending between the first portion 12a and the distal end 24. In some cases, the second portion 12b may at least partially define a closable lumen 26 through the device 12 from the proximal end 22 to the distal end 24.

The first portion 12a of device 12 may be configured to engage the body tissue 16 of the body part 17 (e.g., the first portion 12a of the device 12 may be positioned within a body cavity or passage of the body part 17) at which the defect 14 is located. In some cases, the first portion 12a of the device 12 may be an expanded disk or flange or have disk-like or flange-like shape that may act as a barrier that prevents fluid or material within the body part 17 from passing through the defect 14 when the device 12 is placed at or within the defect 14. In some examples, the first portion 12a of the device 12 may extend radially beyond a circumference of the first opening 14a of the defect 14 and engage the body tissue 16 radially outward from the defect 14. Although not depicted in FIG. 2, the first portion 12a may include one or more tissue engaging elements configured to engage the body tissue 16 and couple the device 12 to the body tissue 16. The tissue engaging elements of the first portion 12a of the device 12 may include, but are not limited to, hooks, barbs, sharp strands, tines, and/or other suitable components configured to engage the body tissue 16.

The first portion 12a of the device 12 may have a configuration that acts as a barrier and blocks fluid or material from entering the defect 14 when the device 12 is implanted in or positioned at the defect 14. To facilitate acting as or being a barrier, the first portion 12a may be or include a dense interwoven wire structure, dense struts, a coating, additional sheet or sheet-like material applied to the wire or tube structure, and/or other suitable components that may act as a barrier to fluid and/or other material flow.

The second portion 12b of the device 12 may be elongated (e.g., relative to the first portion 12a) and may generally have a smaller diameter than the first portion 12a, as depicted for example in FIG. 2. The elongated second portion 12b of the device 12 may be configured to extend an entire length of or at least part of a length of the defect 14. In some examples, the second portion 12b may not be elongated relative to the first portion 12a, but instead have a same length or shorter length than the first portion 12a of the device 12.

The second portion 12b of the device 12 may be tubular. When the second portion 12b is tubular, an inner and/or outer diameter of the second portion 12b may be reduced to selectively close the lumen 26 of the tubular second portion 12b of the device 12.

The second portion 12b of the device 12 may flare radially outward at the distal end 24, but this is not required. When included, the flared distal end 24 may be configured to seal and/or facilitate sealing the second opening 14b of the defect 14 by extending to or beyond a circumference of the second opening 14b.

The second portion 12b of the device 12 may be configured for insertion into the defect 14 and may include an outer surface configured to abut a surface of the defect 14. In some cases, the second portion 12b may include one or more tissue engaging elements 28 configured to engage the surface of the defect 14 when the second portion 12b of the device 12 is positioned within the defect 14. The tissue engaging elements 28 of the first portion 12a of the device 12 may include, but are not limited to, hooks, barbs, sharp strands, tines, and/or other suitable components configured to engage tissue of the defect 14. In some examples, the tissue engaging elements 28 may extend from the second portion 12b of the device 12 such that the tissue engaging elements 28 engage tissue of the defect 14 to prevent or mitigate pull-out of the device 12 in the proximal direction. Further, in some cases, the tissue engaging elements 28 may be configured to engage the tissue of the defect 14 and reduce a diameter of or close the defect 14 as a diameter of the second portion 12b is reduced, which is discussed further below. The tissue engaging elements 28 of or extending from the second portion 12b may extend from a distal end or region of the second portion 12b, extend from one or more longitudinal and/or circumferential portions of the second portion 12b, and/or extend from a plurality of or all locations along the second portion 12b of the device 12.

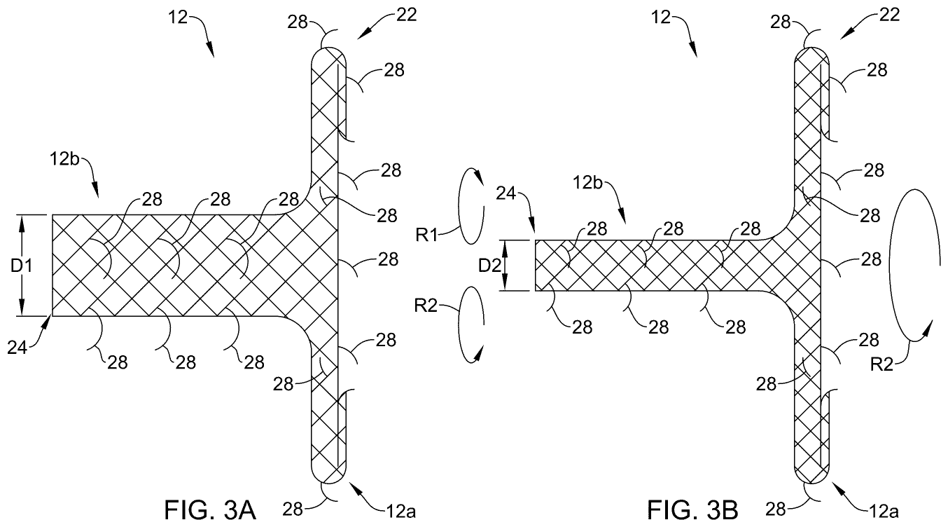

FIGS. 3A and 3B depict an illustrative device 12 for implantation in the defect 14 at or through the body tissue 16. The device 12 depicted in FIGS. 3A and 3B may be configured to reduce a diameter of second portion 12b of the device 12 in response to rotation of the first portion 12a in the direction of arrow R2.

The device 12 of FIGS. 3A and 3B may be formed similar to the other configurations of the device 12 discussed herein. In one example, the device 12 may be formed from one or more interwoven wires, but this is not required.

To facilitate engaging the tissue of the defect 14, the first portion 12a and/or the second portion 12b of the device 12 may include one or more tissue engaging elements 28. As depicted in FIGS. 3A and 3B, the tissue engaging elements 28 extending from the first portion 12a of the device 12 may extend in a first circumferential or rotational direction R1 and the tissue engaging features of the second portion 12b of the device 12 may extend in a second circumferential or rotational direction R2, which may be opposite or substantially opposite the first rotational direction R1, but this is not required. In some cases, one or more of the tissue engaging elements 28 of the first portion 12a and/or the second portion 12b may extend at least partially in a proximal or distal longitudinal direction.

FIG. 3A depicts the device 12 in a deployed configuration with the first portion 12a and the second portion 12b expanded. When expanded in the deployed configuration, the second portion 12b of the device 12 may have a diameter D1.

To reduce the diameter of the second portion 12b of the device 12 depicted in FIG. 3A, the first portion 12a of the device 12 may be rotated to apply a torsional twisting motion to the device 12. When the second portion 12b of the device is positioned at or within the defect 14 and the first portion 12a is rotated in the second rotational direction R2, the tissue engaging elements 28 of or extending from the second portion 12b of the device in the second rotational direction R2 may engage tissue of the defect 14 and pull the walls of the defect radially inward to close the defect 14 as the diameter of second portion 12b is reduced to a diameter D2 in response to the rotation of the first portion 12a relative to the second portion 12b.

As discussed above, the tissue engaging elements 28 of or extending from the first portion 12a of the device 12 may extend in the first rotational direction R1 opposite the second rotational direction R2 (e.g., the tissue engaging elements 28 of the first portion 12a may be counter-angled) relative to the tissue engaging elements 28 of the second portion 12b. As such, when the first portion 12a of the device 12 is rotated and a torsional twisting is applied to the device 12, the tissue engaging elements 28 extending from the first portion 12a may be rotated in the second rotational direction R2 without engaging tissue and then engage the body tissue 16 when the rotation stops or rotational force in the second rotational direction R2 is removed from the device 12 to prevent the device 12 (e.g., the first portion 12a and/or the second portion 12b) from untwisting or unwinding due to energy stored in the wound or twisted device 12 (e.g., as the device 12 may be biased to the deployed configuration) and to maintain the second portion 12b in the closed configuration. When the tissue engaging elements 28 are so configured on the first portion 12a and the second portion 12b of the device 12, the second portion 12b may be reduced in diameter using a ratcheting technique, where the first portion 12a may repeatedly be rotated and then released until the device 12 is adjusted from the deployed configuration to a sufficient closed configuration that results in closing or blocking the defect 14.

Although FIG. 3B depicts the second portion 12b of the device 12 with a constant diameter, it is contemplated that only a portion of the elongated second portion 12b may be configured to be reduced in diameter in response to twisting or rotating of the first portion 12a of the device 12. For example, the second portion 12b of the device 12 may include one or more regions of less dense structure (e.g., one or more axial regions that have a desired pitch of wires or strands, a desired thickness of wires or strands, a desired wall-thickness (e.g., via different coatings applied to wires or strands, different thickness of coatings applied to wires or strands, areas coated versus areas uncoated, etc.), etc.) for reducing a diameter in response to rotation of the first portion 12a.

Rotation of the device 12 may be accomplished in any suitable manner. For example, rotation of the device 12 may be accomplished utilizing a tool that engages the device 12 (e.g., engages the first portion 12a of the device 12), a portion of the delivery components (e.g., the deployment device 20, etc.) that engages at least the first portion 12a of the device (e.g., the device 12 may remain within the elongate tube 18 during rotation of the device 12), and/or via other suitable components.

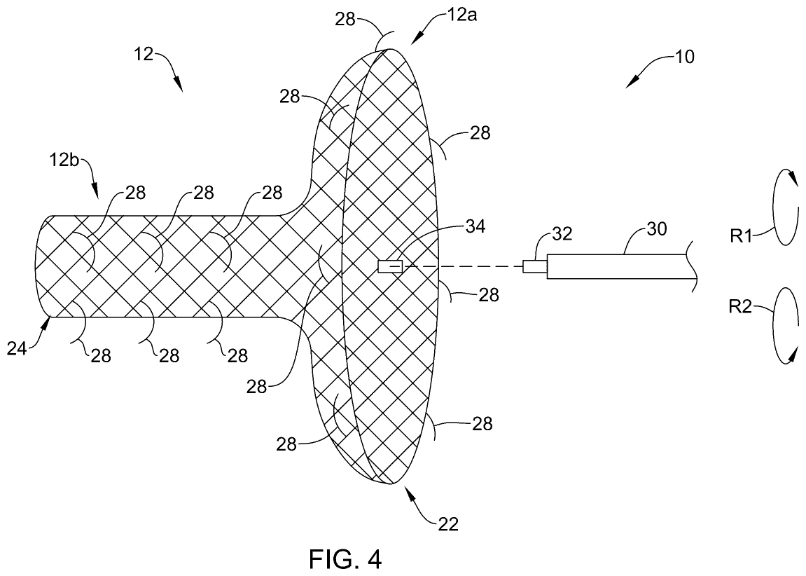

FIG. 4 schematically depicts an illustrative configuration of the system 10 with the device 12 for implanting in the defect 14 at or through body tissue 16 of a body part 17 and a tool 30 for engaging and rotating the first portion 12a of the device 12. In some examples, the tool 30 may be considered to be part of the device 12, but this is not required. The device 12 depicted in FIG. 4 may be configured to be adjusted to rotate the first portion 12a in the direction of arrow R2 and in response, reduce a diameter of second portion 12b of the device 12.

The device 12 of FIG. 4 may be formed similar to the other configurations of the device 12 discussed herein. In one example, the device 12 may be formed from one or more interwoven wires, but this is not required.

To facilitate engaging the tissue of the defect 14, the first portion 12a and/or the second portion 12b of the device 12 may include one or more tissue engaging elements 28. As depicted in FIG. 4, the tissue engaging elements 28 extending from the first portion 12a of the device 12 may extend in the first rotational direction R1 and the tissue engaging features 28 of the second portion 12b of the device 12 may extend in a second rotational direction R2, which may be opposite or substantially opposite the first rotational direction R1, but this is not required. In some cases, one or more of the tissue engaging elements 28 of the first portion 12a and/or the second portion 12b may extend at least partially in a proximal and/or distal longitudinal direction.

The tool 30 may be elongated and include a first keyed portion 32 at a distal end of the tool 30. The first keyed portion 32 of the tool may be configured to mate with a second keyed portion 34 at the first portion 12a of the device 12. In some examples, the second keyed portion 34 may be aligned with a central longitudinal axis of the device 12, but this is not required.

The first keyed portion 32 and the second keyed portion 34 may take on any suitable configuration configured to rotate the device 12 in response to rotation of the tool 30 when the first keyed portion 32 and the second keyed portion 34 are mating. For example, one of the first keyed portion 32 and the second keyed portion 34 may be a male component and the other of the first keyed portion 32 and the second keyed portion 34 may be a female component (e.g., an opening). The first and second keyed portions 32, 34 may have mating shapes including, but not limited to, rectangles, squares, ovals, stars, and/or other suitable shapes that facilitate rotating the device 12 in response to rotation of the tool 30. In some examples, the first keyed portion 32 may be a male component, the second keyed portion 34 may be a female component, and the first and second keyed portions 32, 34 may be rectangular shaped, as depicted in FIG. 4, but other suitable configurations are contemplated.

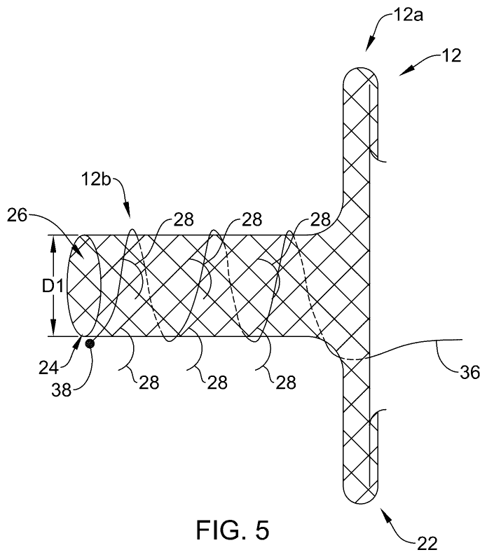

FIGS. 5-7 schematically depict an illustrative configuration of the device 12 for implanting in the defect 14 at or through body tissue 16 of a body part 17. The device 12 depicted in FIGS. 5-7 may be configured to reduce a diameter of the second portion 12b of the device 12 in response to applying a tension to a cord 36 (e.g., a suture, a thread, a wire, etc.) wrapped around the second portion 12b of the device 12 and extending proximal of the first portion 12a.

The device 12 of FIGS. 5-7 may be formed similar to the other configurations of the device 12 discussed herein. In one example, the device 12 may be formed from one or more interwoven wires, but this is not required.

To facilitate engaging the tissue of the defect 14, the first portion 12a and/or the second portion 12b of the device 12 may include one or more tissue engaging elements 28. As depicted in FIGS. 5-7, the second portion 12b of the device 12 may include tissue engaging elements 28 extending in a circumferential or rotational direction. In some cases, one or more of the tissue engaging elements 28 may extend at least partially in a proximal and/or distal longitudinal direction.

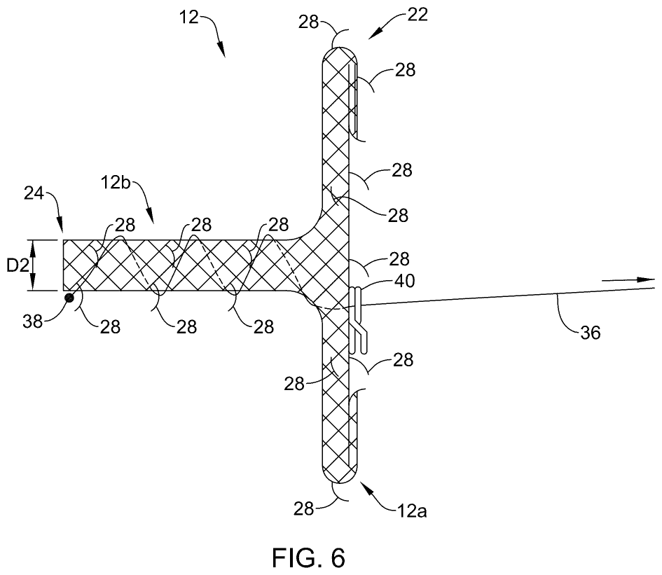

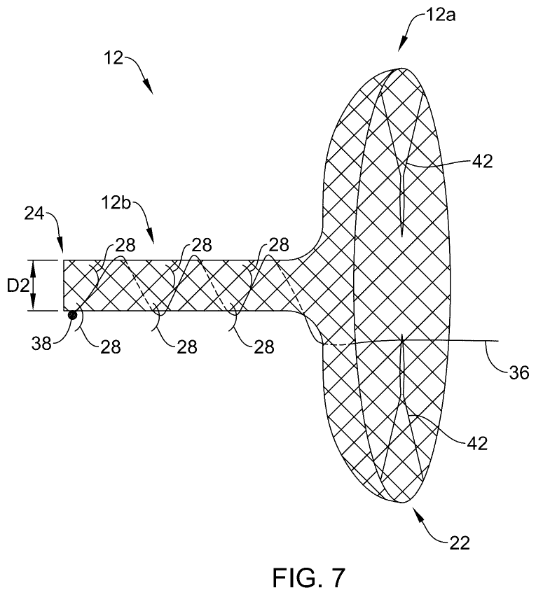

The cord 36 may be wrapped around (e.g., helically wrapped around and/or otherwise wrapped around) the second portion 12b of the device 12. In some examples, the cord 36 may be encapsulated within, raveled about, threaded through, and/or otherwise wrapped around or along the wires or struts of the second portion 12b such that when a tension or proximal force is applied to the cord 36, the second portion 12b of the device 12 is collapsed or reduced in diameter from the expanded diameter D1 (e.g., as depicted in FIG. 5) of the device 12 in an expanded configuration to a reduced diameter D2 (e.g., as depicted in FIGS. 6 and 7) of a closed configuration of the device 12.

In some cases, a distal end of the cord 36 may be coupled to or relative to the second portion 12b of the device 12 and/or may include an anchor or a stop 38 configured to prevent the cord 36 from being fully pulled through and out of the second portion 12b of the device 12. The stop 38 may be any suitable component configured to couple the cord 36 with the second portion 12b of the device 12 including, but not limited to, an adhesive, a knot, a ball, and/or other suitable stop component configured to engage the wire, strut, layered material, and/or other components of the distal end 24 of the second portion 12b.

In response to a proximal force applied to the cord 36, the cord 36 may radially tighten and the second portion 12b of the device 12 may compress to a closed configuration having a reduced diameter D2. For example, when a proximal force is applied to the cord 36, a tension is applied to the cord 36, the stop 38 engages a surface or structure of the second portion 12b of the device 12, and the portion of the cord 36 wrapped around the second portion 12b squeezes or compresses the second portion 12b to adjust the device 12 to the closed configuration. When the second portion 12b is located within the defect 14, the tissue engaging elements 28 may engage the wall tissue of the defect 14 and draw the wall radially inward as the second portion 12b adjusts to the closed configuration in response to a tension being applied to cord 36.

One or more components may be utilized to maintain a tension on the cord 36 and prevent the device 12 from adjusting to the expanded configuration from the closed configuration. Example suitable components for maintaining a tension on the cord 36 include, but are not limited to, clips, wedges, and/or other suitable components configured to grasp the cord 36 and maintain a tensioned applied thereto relative to the device 12.

FIG. 6 depicts the device 12 with a tension applied to the cord 36 in a proximal direction (e.g., as indicted by the arrow pointing in the proximal direction) to reduce the diameter of the second portion 12b to the diameter D2 associated with the closed configuration of the device 12. In some examples, a clip 40 may be applied to the cord 36 at a location proximate to where the cord 36 proximally exits first portion 12a of the device 12 such that the clip 40 is coupled to the cord 36 and the wires or struts of the first portion 12a and/or the clip 40 is coupled to the cord 36 at a location abutting a proximal surface of the first portion 12a to maintain the tension applied to the cord 36 and prevent the second portion 12b from expanding to the expanded configuration. Once the clip 40 has been applied to the cord 36 at or proximate the first portion 12a of the device 12, the portion of the cord 36 proximal of the clip 40 may be separated from the portion of the cord 36 at or distal of the clip 40, but this is not required.

The clip 40 may be any suitable type of clip that can be placed at or adjusted to a location proximate the first portion 12a when the tension is applied to the cord 36 and secured relative to the device 12 (e.g., secured so as not to pass through the structure (e.g., wires, struts, strands, etc.) of the device 12) to maintain the tension on the cord 36. Example suitable types of clips 40 may include, but are not limited to, vessel clips, ligation clips, spring clips, magnetic clips, and/or other suitable types of clips.

FIG. 7 depicts the device 12 with a tension applied to the cord 36 in the proximal direction (e.g., as indicted by the arrow pointing in the proximal direction) to reduce the diameter of the second portion 12b to the diameter D2 associated with the closed configuration of the device 12. In some examples, one or more wedges 42 may be formed at or attached to the first portion 12a of the device 12. The one or more wedges 42 may be located at a proximal side or end of the first portion 12a such that a tensioned cord 36 may be inserted into the wedge 42 and secured therein through a friction fit or other type of connection that maintains the tension applied to the cord 36 and prevents the second portion 12b from expanding to the expanded configuration. As depicted in FIG. 7, the device 12 may include two wedges opposing one another, but a single wedge 42 or more than two wedges may be utilized, as desired. When multiple wedges 42 are utilized, the cord 36 may be threaded into and/or secured within multiple wedges to redundantly secure the cord 36 relative to the first portion 12a of the device 12. Once the one or more wedges 42 have received and secured the cord 36, the portion of the cord 36 proximal of the wedge 42 may be separated from the portion of the cord 36 at or distal of the wedge 42, but this is not required.

The wedge 42 may be any suitable type of wedge that is configured to receive the cord 36 and secure the cord 36 therein. In some examples, the wedge 42 may include a v-shape, a substantially v-shape, or other suitable shape such that the cord 36 may be slid or threaded into the apex of the wedge 42. Once the cord 36 is at the apex of the wedge 42, the cord 36 may be unable to slide relative to the wedge 42 and/or the first portion 12a of the device 12 and tension on the cord 36 may be maintained.

The one or more wedges 42 may be formed in any suitable manner. In some examples, the wedges 42 may be formed from the wire or struts forming the device 12, but this is not required. Additionally or alternatively, one or more wedges 42 may be welded or otherwise secured to the wire or struts forming the device 12.

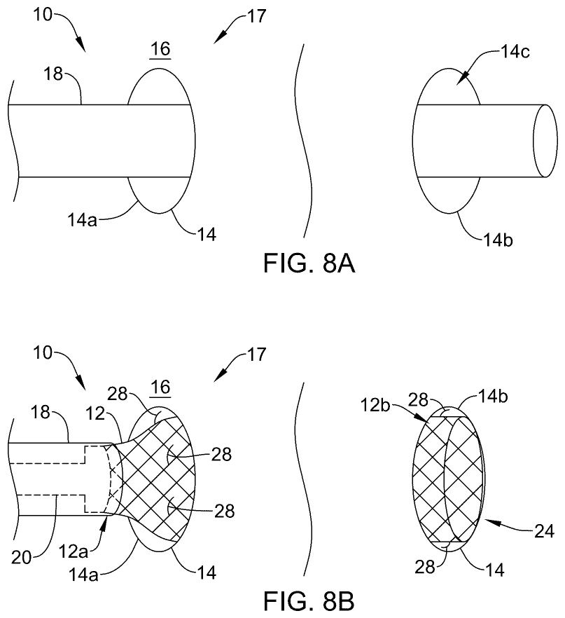

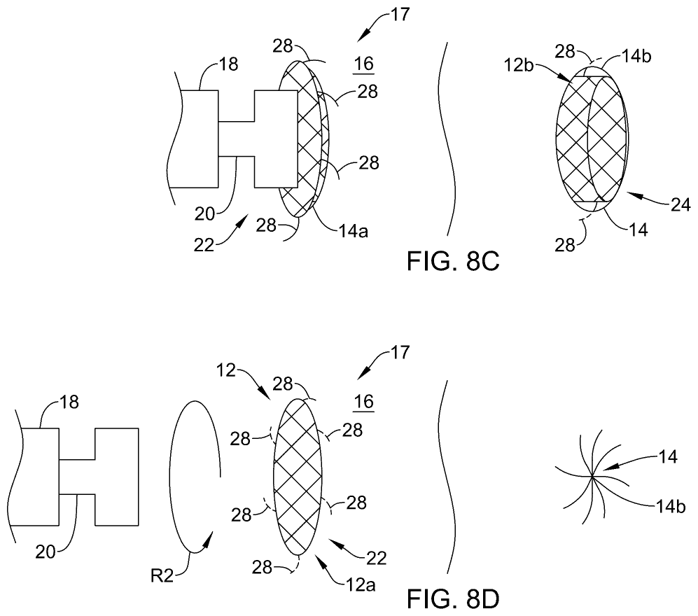

FIGS. 8A-8D schematically depict an illustrative technique for applying the device 12 to a defect 14 in body tissue 16 of a body part 17, where the left side of each figure is a view from within a cavity or passage of the body part 17 and the right side of each figure is a view from exterior of the body part 17. As depicted in FIGS. 8A-8D, the defect 14 extends through the body tissue 16 and includes a first opening 14a, a second opening 14b, and a passage 14c extending between the first opening 14a and the second opening 14b.

As depicted in FIG. 8A, the elongate tube 18 may be inserted into the defect 14 (e.g., into the passage 14c of the defect 14). The elongate tube 18 may be delivered to the defect 14 endoscopically and/or in one or more other suitable manners.

FIG. 8B schematically depicts the positioning and the deployment of the device 12 to a partially deployed or expanded configuration within the defect 14, where the second portion 12b is positioned and expanded within the defect 14, while first portion 12a of the device 12 may remain within the elongate tube 18. In some cases, the elongate tube 18 may be withdrawn to deploy the second portion 12b of the device 12 and/or the deployment device 20 may be advanced relative to the elongate tube 18 to push the second portion 12b out of a distal end of the elongate tube 18.

FIG. 8C schematic depicts the device 12 deployed at the defect 14. In some cases, the device 12 may be partially rotated or otherwise adjusted once the second portion 12b is within the defect 14 such that the tissue engaging elements 28 initially engage tissue of the defect 14, as depicted in FIG. 8C (e.g., as represented by a portion of the tissue engaging elements 28 being shown in broken lines). The partial rotation or other adjustment of the device 12 may be accomplished by using the elongate tube 18, using the deployment device 20, using the tool 30 (not shown in FIGS. 8A-8D), applying tension to a cord 36 (not shown in FIGS. 8A-8D), and/or using one or more other suitable components configured to engage the device 12 and facilitate adjustment of the device 12 within the defect 14.

Although the device 12 has been rotated or otherwise adjusted such that the tissue engaging elements 28 of the second portion 12b of the device 12 are engaging the tissue of the defect 14, the tissue engaging elements 28 of the first portion 12a, when included, may or may not (e.g., as depicted in FIG. 8C) engage the body tissue 16. In some cases, the first portion 12a of the device 12 may remain within the elongate tube 18 while the device 12 is initially rotated or adjusted such that the tissue engaging elements 28 of the second portion 12b engage tissue of the defect 14.

After the tissue engaging elements 28 of the second portion are engaging tissue of the defect 14, the device 12 may be further rotated or adjusted (e.g., via the elongate tube 18, the deployment device 20, the tool 30, the cord 36, and/or other suitable component(s)) to reduce the diameter of the second portion 12b and close the defect 14. In one example, the deployment device 20 may be actuated to advance the device 12 (e.g., the first portion 12a and/or the second portion 12b) out of the elongate tube 18 to a deployed configuration, as depicted in FIG. 8C, and grasp the first portion 12a of the device 12 to rotate the device 12 and reduce a diameter of the second portion 12b. In another example, the deployment device 20 may be actuated to advance the device 12 (e.g., the first portion 12a and/or the second portion 12b) out of the elongate tube 18 to a deployed configuration, grasp the cord 36, and apply a tension to the cord 36 to reduce a diameter of the second portion 12b.

FIG. 8D depicts the device 12 within the defect 14 and in the closed configuration, with the deployment device 20 and the elongate tube 18 removed from the first portion 12a. In the closed configuration, the device 12 has been rotated in the direction of arrow R2 or otherwise adjusted to reduce the diameter of the second portion 12b from a diameter associated with the expanded configuration to a diameter associated with the closed configuration. As depicted on the right side of FIG. 8D, the second opening 14b of the defect 14 has been closed. As depicted on the left side of FIG. 8D, the tissue engaging elements 28 of the first portion 12a of the device 12 may engage the body tissue 16 (e.g., as represented by the tissue engaging elements 28 being shown in broken lines) to prevent the device 12 from unwinding and adjusted from the closed configuration to the expanded configuration.

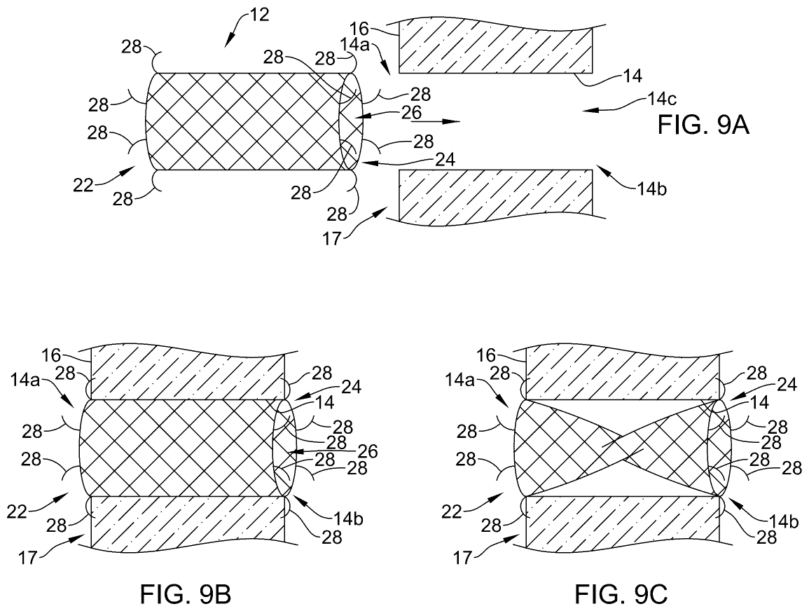

FIGS. 9A-9C depict a schematic positioning and implantation of an illustrative configuration of the device 12 at or through a defect 14 in body tissue 16 of a body part 17 (e.g., a fistula extending through tissue of a body part along the GI tract and/or other defect). The device 12 depicted in FIGS. 9A-9C may be configured to be adjusted to close the lumen 26 of the device 12 after being positioned within the defect 14 to occlude the defect 14.

The device 12 of FIGS. 9A-9C may be formed similar to the other configurations of the device 12 discussed herein. In one example, the device 12 may be formed from one or more interwoven wires, but this is not required. Further, the body of the device 12 depicted in FIGS. 9A-9C may be configured to prevent fluid and/or other material from passing therethrough, such that when the device 12 within the defect 14 is adjusted to close the lumen 26 through the device, the body of the device 12 occludes the defect 14.

Tissue engaging elements 28 may extend from the proximal end 22 of a body of the device 12 and/or the distal end 24 of the body of the device 12 to engage tissue proximate the first opening 14a and/or the second opening 14b of the defect 14. Additionally or alternatively, the tissue engaging elements 28 may be located at other locations along the device 12 to engage tissue of the defect 14 and/or tissue proximate the defect, as desired.

FIG. 9A depicts the device 12 aligned with the defect 14 extending through the body tissue 16 of the body part 17. The device 12 may be advanced into the defect 14 in the direction of arrow A until the distal end 24 of the device 12 is proximate the second opening 14b of the defect. In some cases, the device 12 may be configured to contact the walls of the passage 14c of the defect 14 such that fluid and/or other material passing through the defect 14 while the device 12 is placed in the defect 14 passes through the lumen 26 of the device 12. Although not depicted, the device 12 may be initially positioned within defect 14 while in an elongate tube or other suitable delivery mechanism.

Once the distal end 24 of the device 12 is positioned proximate the second opening 14b of the defect 14, the tissue engaging elements 28 at the distal end 24 of device 12 may engage with the tissue proximate the second opening 14b of the defect 14, as depicted for example in FIG. 9B. In some examples, a user may manipulate or adjust (e.g., adjust laterally, rotationally, etc.) the device 12 to cause the tissue engaging elements 28 to engage tissue proximate the second opening 14b of the defect 14. In one example, a user may extend the device 12 through the defect 14 such that the distal end 24 of the device 12 extends through the second opening 14b and then pull-back on the device 12 to engage the tissue engaging elements 28 with the tissue proximate the second opening 14b. When so positioned, the proximal end 22 of the device 12 may extend proximally out of the first opening 14a of the defect 14, as depicted for example in FIG. 9B, but this is not required.

Once the tissue engaging elements 28 at the distal end 24 of the device 12 are engaging tissue proximate the second opening 14b of the defect 14 and/or tissue of the defect 14 to secure the distal end 24 relative to the defect 14, the proximal end 22 of the device 12 may be rotated. Rotation of the proximal end 22 of the device 12 while the distal end 24 is secured relative to the defect 14, may result in a central portion 44 of the device 12 twisting and closing the lumen 26, which reduces a diameter of the device 12 at the central portion 44, as depicted in FIG. 9C.

As the device 12 twists, a length of the device 12 may shorten such that the tissue engaging elements 28 at the proximal end 22 of the device 12 are positioned proximate the first opening 14a of the defect 14 or other suitable location along the defect 14. Once the tissue engaging elements 28 at the proximal end 22 of the device 12 are proximate the first opening 14a of the defect 14, the tissue engaging elements 28 may engage the body tissue 16 and/or tissue of the defect 14 proximate the first opening 14a and/or other tissue of the defect 14 to maintain the device 12 in a twisted configuration occluding the defect 14.

One or more of the tissue engaging elements 28 at the proximal end 22 of the device 12 may be proactively engaged with the body tissue 16 or tissue of the defect 14 and/or one or more of the tissue engaging elements 28 may automatically engage the body tissue 16 and/or other tissue of the defect 14 when a rotational force twisting the device 12 is removed such that the tissue engaging elements 28 prevent the device 12 from untwisting. As discussed above, the tissue engaging elements 28 located at the proximal end 22 of the device 12 may extend radially outward from the body of the device 12 in a circumferential direction opposite a direction of the rotational force applied to the device 12 to twist the device 12 and occlude the lumen 26 and the defect 14, but this is not required and other suitable configurations are contemplated.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A device for implantation at an anatomical defect in or through body tissue, the device comprising:
a first portion configured to engage the body tissue; and
a second portion extending distally from the first portion, the second portion is configured to engage tissue of the anatomical defect, the anatomical defect having a first opening, a second opening, and a passage extending between the first opening and the second opening, and
wherein the second portion is configured to be reduced in diameter while engaging the tissue of the anatomical defect to close the anatomical defect.

2. The device of claim 1, wherein the second portion is tubular.

3. The device of claim 1, further comprising:
one or more barbs extending from the second portion, the one or more barbs are configured to engage the tissue of the anatomical defect.

4. The device of claim 3, wherein the one or more barbs extend in a circumferential direction around the second portion and are configured to engage the tissue of the anatomical defect in response to rotation of the second portion.

5. The device of claim 1, further comprising:
a first set of one or more barbs extending from the first portion in a first circumferential direction, the first set of one or more barbs are configured to engage the body tissue; and
a second set of one or more barbs extending from the second portion in a second circumferential direction, the second set of one or more barbs are configured to engage the tissue of the anatomical defect.

6. The device of claim 5, wherein:
the second set of one or more barbs is configured to engage the tissue of the anatomical defect in response to rotation of the second portion in the second circumferential direction, and
while engaging the tissue of the anatomical defect, the second portion is configured to be reduced in diameter to close the anatomical defect in response to rotation of the first portion in the second circumferential direction.

7. The device of claim 6, wherein the first set of one or more barbs are configured to engage the body tissue to prevent rotation of the first portion and the second portion in the first circumferential direction.

8. The device of claim 1, further comprising:
a first set of one or more barbs extending from the first portion, the first set of one or more barbs are configured to engage the body tissue; and
a second set of one or more barbs extending from the second portion, the second set of one or more barbs are configured to engage tissue distal of the anatomical defect.

9. The device of claim 1, further comprising:
a tool for engaging the first portion, and
wherein the first portion has an opening for receiving a distal end of the tool and the tool is configured to be rotated when received within the opening to reduce the diameter of the second portion.

10. The device of claim 1, further comprising:
a cord wrapped about the second portion and extending proximal of the first portion, and
wherein the cord causes the second portion to be reduced in diameter in response to a tension applied to the cord.

11. The device of claim 10, further comprising:
a clip, and
wherein the clip is configured to be secured to the cord at a location proximate the first portion when a tension is applied to the cord to maintain the tension on the cord.

12. The device of claim 10, wherein the first portion includes a wedge configured to receive the cord after the tension is applied to the cord and maintain the tension on the cord.

13. The device of claim 1, wherein the first portion and the second portion are formed from one or more interwoven wires.

14. A system for implanting an implantable device in an anatomical defect at or through body tissue, the system comprising:
an elongate tube configured for insertion to the body tissue proximate the anatomical defect;
a deployment device configured to be received within the elongate tube; and
an implantable medical device configured to be received within the elongate tube and engage the deployment device, the implantable medical device comprising:
a first portion configured to engage the body tissue; and
a second portion extending distally from the first portion, the second portion is configured to engage tissue of the anatomical defect, the anatomical defect having a first opening, a second opening, and a passage extending between the first opening and the second opening, and
wherein the deployment device is configured to be actuated to advance the second portion of the implantable medical device out of the elongate tube to a deployed configuration having a first diameter within the anatomical defect and reduce a diameter of the second portion of the implantable medical device within the anatomical defect to a second diameter.

15. The system of claim 14, wherein the deployment device is configured to engage and rotate the first portion of the implantable medical device to reduce the diameter of the second portion of the implantable device.

16. The system of claim 14, wherein:
the implantable medical device comprises a cord wrapped about the second portion; and
the deployment device is configured to engage the cord and apply a tension to the cord to reduce the diameter of the second portion of the implantable medical device to the second diameter.

17. A method of implanting an implantable medical device in an anatomical defect at or through body tissue, the method comprising:
positioning an elongate portion of the implantable medical device within the anatomical defect;
closing the anatomical defect in response to reducing a diameter of the implantable medical device within the anatomical defect from a first diameter to a second diameter; and
securing the implantable medical device at or proximate the anatomical defect in a configuration having the second diameter.

18. The method of claim 17, wherein closing the anatomical defect in response to reducing the diameter of the elongate portion comprises engaging one or more barbs extending from the elongate portion of the implantable medical device with tissue of or proximate the anatomical defect.

19. The method of claim 18, wherein closing the anatomical defect in response to reducing the diameter of the elongate portion comprises applying a tension to a cord wrapped about the elongate portion.

20. The method of claim 17, wherein securing the implantable medical device at or proximate the anatomical defect in the configuration having the second diameter comprises engaging barbs extending from the implantable medical device with the body tissue.
